(19) United States
(12) Patent Application Publication
(10) Pub. No.: US 2009/0326415 A1
(43) Pub. Date: Dec. 31, 2009

(54) MICRONEEDLES AND METHODS FOR FABRICATING MICRONEEDLES

(75) Inventor: Chee Yen Lim, Singapore (SG)

Correspondence Address: FOLEY AND LARDNER LLP;SUITE 500, 3000 K STREET NW, WASHINGTON, DC, 20007, US

(73) Assignee: AGENCY FOR SCIENCE ,TECHNOLOGY AND RESEARCH

(21) Appl. No.: 12/439,142

(22) PCT Filed: Aug. 28, 2006
(86) PCT No.: PCT/SG2006/000246

§ 371 Date: May 14, 2009

(51) Int. Cl. A61B 5/15; B29C 33/40; B29C 45/00; A61M 5/00

(52) U.S. Cl. 600/573; 264/219; 264/328.1; 604/272; 604/173

(57) ABSTRACT

A plastic microneedle comprising: a body portion tapering from a larger end of the body portion towards a tip portion of the body portion; at least one side port; and a lumen extending from the larger end of the body portion and within the body portion of the microneedle, wherein the side port extends into the lumen such that the side port and the lumen are in fluid communication with each other.

### FIELD OF INVENTION

The present invention relates broadly to microneedles, and microneedle structures, and methods for fabricating microneedles, and microneedle structures.

### BACKGROUND

Microstructures such as microneedles are being utilised in various technology fields, for example, in the administration of drugs to a body. Drugs can be administered to the body by, for example, injection. A typical injection operation involves a syringe with a needle which is first used to breach the skin and then to be inserted to reach a desired depth in the body before the drug is injected into the body. There are various modes of delivery of drugs into the body, for example, transdermal delivery which delivers drugs to different parts of the body within an epidermis layer of the skin. Generally, microneedles should be sharp and robust enough to pierce the skin for the administration of drugs into the body.

Conventional microneedles are made of, for example, silicon (Si), plastic or metal. Known plastic microneedles typically have limited sharpness at their tips and are generally not strong enough for penetrating the skin. One known method of fabricating plastic microneedles involves using the inclined LIGA (Lithografie, Galvanoformung, Abformung) process. One drawback of this method is the high costs of the facilities required as well as the use of expensive photosensitive materials.

Silicon microneedles are typically fabricated using silicon wafer as the raw material and employing wafer fabrication technology which requires a clean-room environment. One disadvantage of silicon microneedles is the high capital investment required for setting up and maintaining clean-room and wafer fabrication facilities and high production costs which render silicon microneedles unsuitable as single-use, disposable products.

Metal microneedles can typically be fabricated by metallization of Si or plastic moulds followed by a releasing process in which the mould is dissolved. Therefore, new moulds must be created for each metal microneedle fabrication process.

Further, known methods of fabricating microneedles typically involve the use of customised or specialised equipment that are not cost effective and not feasible for mass production of microneedles.

Therefore, there is a need for microneedles and methods and apparatus for fabricating microneedles that seek to address or overcome at least one of the abovementioned problems.

### SUMMARY

According to a first aspect of the present invention, there is provided a plastic microneedle comprising: a body portion tapering from a larger end of the body portion towards a tip portion of the body portion; at least one side port; and a lumen extending from the larger end of the body portion and within the body portion of the microneedle, wherein the side port extends into the lumen such that the side port and the lumen are in fluid communication with each other.

The side port may be disposed on a side surface of the body portion of the microneedle.

The microneedle may comprise two side ports disposed at opposing side surfaces of the body portion of the microneedle.

The microneedle may further comprise an extended tip portion formed on the tip portion of the body portion and forming an apex of the microneedle, wherein the extended tip portion comprises a substantially elongate protrusion extending from the tip portion.

A geometry of the extended tip portion may be substantially the same or different from a geometry of the body portion.

The microneedle may be made from at least one of a group of polymers consisting of polycarbonate (PC), polystyrene (PS), polyetherimide (PEI), polyetherehterketone (PEEK).

The body portion of the microneedle may be pyramid shaped, conical shaped, hexagonal shaped, etc.

The microneedle may be fabricated using injection moulding or cast moulding.

According to a second aspect of the present invention, there is provided a platform member comprising an array of microneedles.

According to a third aspect of the present invention, there is provided a method of fabricating a plastic microneedle, the method comprising: forming a body portion, the body portion tapering from a larger end of the body portion a tip portion of the body portion; forming at least one side port; and forming a lumen extending from the larger end of the body portion and within the body portion of the microneedle, wherein the side port extends into the lumen such that the side port and the lumen are in fluid communication with each other.

The method may further comprise: forming an extended tip portion on the tip portion of the body portion and the extended tip portion forming an apex of the microneedle, wherein the extended tip portion comprises a substantially elongate protrusion extending from the tip portion.

The method may comprise: forming a master mould comprising a positive shape of the microneedle, using the master mould to form a secondary mould comprising a cavity comprising a periphery of a negative shape of the microneedle; and using the secondary mould to form the microneedle.

The method may comprise: forming a master mould comprising a positive shape of the microneedle, using the master mould to form an intermediate mould comprising a cavity comprising a periphery of a negative shape of the microneedle; and using the intermediate mould to form a secondary mould comprising a cavity defining the periphery of a negative shape of the microneedle.

The intermediate mould may be made of polymer.

The positive shape of the microneedle on the master mould may be created by precision wire-cutting.

The secondary mould may be made of metal.

The method may further comprise: filling the cavity of the secondary mould with a polymer to form the microneedle.

The method may further comprise: providing an insert member;
providing the secondary mould, the secondary mould further comprising at least one slot portion for receiving and aligning the insert member such that a leading edge of the insert intersects the periphery of the negative shape of the microneedle; inserting the insert member into the secondary mould; and filling the secondary mould with a fill material, wherein the insert creates the lumen in the microneedle and the intersection of the leading edge of the insert with the periphery of the negative shape of the microneedle creates the side port extending into the lumen such that the lumen and the side port are in fluid communication with each other.

The periphery of the negative shape of the microneedle may comprise a channel extending from an apex of the negative shape of the microneedle for forming the extended tip portion of the microneedle.

The method may further comprise filling the channel at least partially to form the extended tip portion.

According to a fourth aspect of the present invention, there is provided a use of a microneedle for injecting liquid into a body.

According to a fifth aspect of the present invention, there is provided a use of a microneedle for extracting body fluid from a body.

Extracting body fluid from the body may include whole blood sampling.

### BRIEF DESCRIPTION OF THE DRAWINGS

Embodiments of the invention will be better understood and readily apparent to one of ordinary skill in the art from the following written description, by way of example only, and in conjunction with the drawings, in which:

FIG. 1(a) is a schematic drawing of a cross sectional view of a microneedle;

FIG. 1(b) is a schematic drawing of the microneedle in FIG. 1 (a) showing dispensing of drugs to the skin;

FIG. 2 is a schematic drawing of a cross sectional view of another microneedle;

FIG. 3(a) is a micrograph of another microneedle;

FIG. 3(b) is a micrograph of a close-up view of the microneedle of FIG. 3(a);

FIGS. 4(a) and 4(b) are schematic drawings of a portion of a microneedle without an extended tip portion and a microneedle with an extended tip portion, respectively;

FIGS. 5(a)-5(d) are schematic drawings of a mould used for fabricating a microneedle;

FIG. 6 is a schematic drawing of a portion of a mould halve used for fabricating microneedles;

FIG. 7 is a schematic drawing of a cross section of another mould halve used for fabricating a microneedle;

FIG. 8 is a schematic drawing of an isometric view of another pin;

FIG. 9 is a schematic drawing of an isometric view of another pin;

FIG. 10 is a schematic drawing of an isometric view of another pin;

FIGS. 11(a)-11(c) are schematic drawings of a process for making a secondary mould for use in fabricating microneedles;

FIGS. 12(a)-12(d) are schematic drawings of another process for making a secondary mould for use in fabricating microneedles;

FIGS. 13(a)-13(c) are schematic drawings of a process for making an intermediate mould;

FIGS. 14(a)-14(c) are schematic drawings of another process for making an intermediate mould;

FIGS. 15(a)-15(d) are schematic drawings of a process for making a secondary mould using an intermediate mould;

FIG. 16(a) is a schematic drawing of another microneedle;

FIG. 16(b) shows a plurality of microneedles of FIG. 16(a) assembled onto a platform to form a microneedle module;

FIG. 17 is a schematic drawing illustrating a transdermal drug delivery mode;

FIG. 18 is a micrograph of an array of microneedles having side ports and an extended tip portion; and

FIGS. 19(a) to 19(c) are schematic drawings of how skin is compressed and tensioned prior to application of microneedles to the skin.

### DETAILED DESCRIPTION

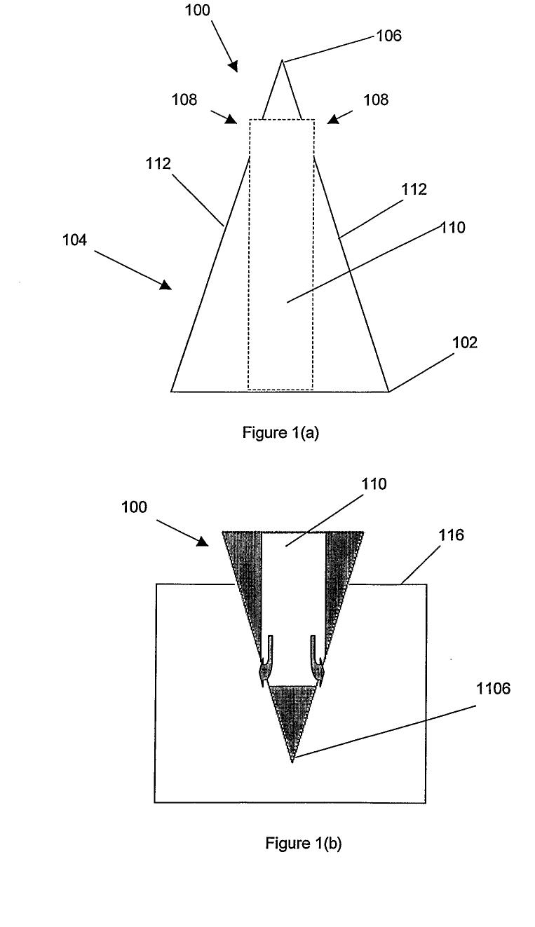

A schematic drawing of a microneedle 100 is shown in FIG. 1(a). The microneedle 100 comprises a body 104, a tip 106, two dispensing ports 108 and a lumen 110 extending within the body 104 of the microneedle 100. The body 104 tapers from a larger end 102 of the body 104 towards the tip 106 of the body 104. The two dispensing ports 108 are located on side portions of the microneedle (hence termed side-ports) and disposed on opposing sidewalls 112 (i.e. opposing surfaces 112 of the body 104 of the microneedle 100), at a specified distance away from the tip 106 of the microneedle 100. The lumen 110 extends within the body 104 of the microneedle 100 from the larger end 102 of the body 104 upwards towards the tip 106 and leads to the two side-ports 108 such that the lumen 110 is in fluid connection with the two side-ports 108. The lumen 110 terminates at the two side-ports 108 leaving a distance between the side ports 108 and the tip 106 of the microneedle 100. A schematic drawing of a microneedle 100 breaching the skin 116 for drug delivery is shown in FIG. 1(b). After the skin 116 is breached by the microneedle 100, a portion of the microneedle 100 comprising at least the tip 106 and the side-ports 108 is inserted into the skin 116. The drug to be dispensed passes through the lumen 110 and is delivered into the skin 116 via the side-ports 108, as indicated by the arrows in FIG. 1(b).

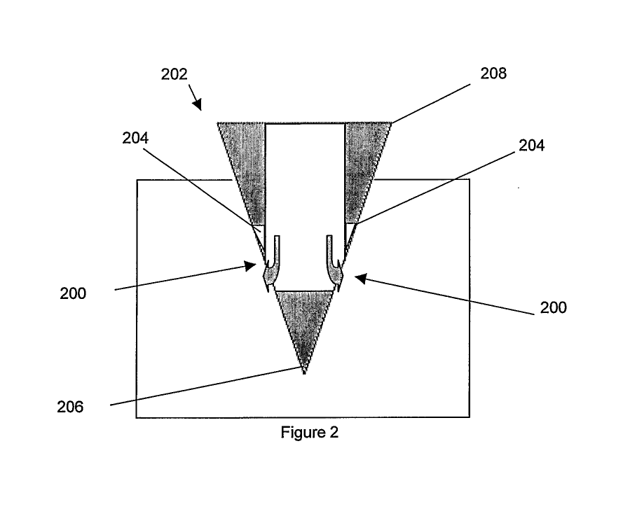

In another embodiment, each side port 200 of the microneedle 202 comprises an inwardly chamfered area 204 at a lower portion of the port 200 away from the tip 206 of the microneedle 202 (i.e. towards the larger end 208 of the body of the microneedle 202) is shown in FIG. 2. The inwardly chamfered areas 204 help to dispense drugs more effectively and minimises clogging of the side ports 200 during penetration of the microneedle 202 into skin. A portion of the port nearer the tip 206 of the microneedle 202 together with the tip 206 of the microneedle serve the purpose of breaching the skin and subsequently expanding and holding the skin in place, so that the drugs can be dispensed through the side ports 200, first into the clearance created by the chamfered areas 204 and then into the skin.

The distance between the side ports and the tip of the microneedle, termed the submerged distance, is chosen such that when the microneedle is used to breach the skin, the distance of the side ports from the tip of the microneedle is sufficiently far enough for the side ports to be isolated from the breaching and compressing action of the skin yet close enough to the tip of the microneedle such that the side-ports can be completely buried within the skin for effective drug dispensing without spillage. Another consideration is that the nearer the side-ports are to the tip of the microneedle, the weaker the mechanical structure of the microneedle is. Depending on the materials used, the geometry of the microneedle and the size and number of the side-ports, the distance between the centre of the side-port from the tip/apex of the microneedle can vary around 300 microns approximately, for example, between about 100-500 microns, for a microneedle of about 1000 microns in length.

In the microneedles described above, each microneedle comprises two side-ports. It should be appreciated that for hollow microneedles (i.e. microneedles with a lumen), for example, the microneedle 100 in FIG. 1(a), at least one side-port (in fluid communication with the lumen) is required to allow dispensation of the drugs. It will be appreciated that the number of side ports required can be varied depending on design requirements.

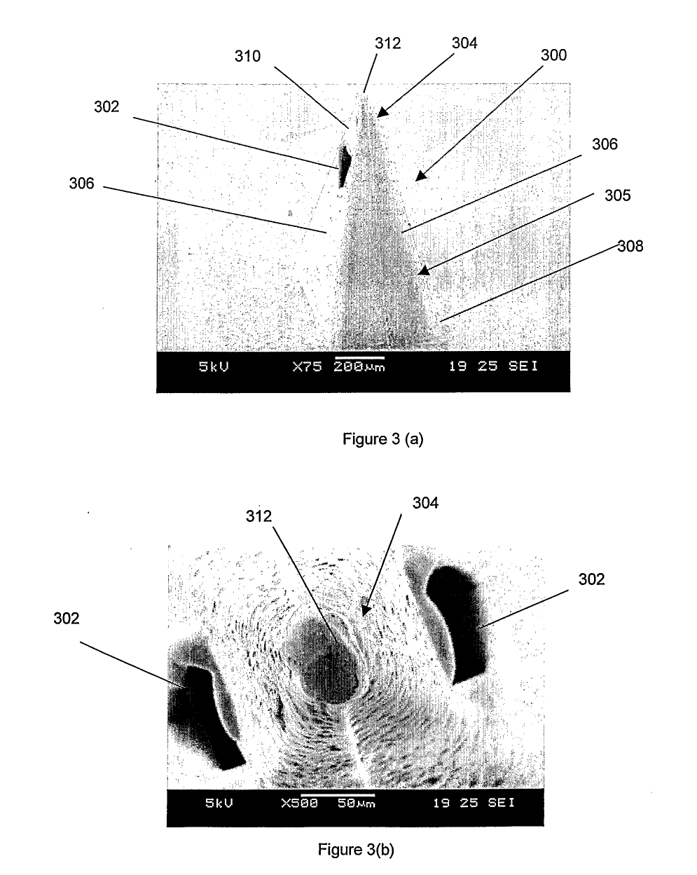

A micrograph of another microneedle 300 is shown in FIG. 3(a). A micrograph of a close-up view of the microneedle 300 of FIG. 3(a) is shown in FIG. 3(b), when the microneedle 300 is viewed from the top. In addition to two side-ports 302, the microneedle 300 comprises an extended tip portion 304. The microneedle 300 is pyramid shaped and the side-ports 302 are located on opposed sidewalls 306 of the body 305. The larger end 308 of the body 305 of the microneedle 300 is about 400 μm and the width of the extended tip portion 304 of the microneedle 300 is about 50 μm. The extended tip portion 304 is formed on the tip 310 of the body 305 and forms an apex 312 of the microneedle 300. The extended tip portion 304 comprises a substantially elongate protrusion extending from the tip portion 310 of the microneedle 300. The extended tip portion 304 forms the apex 312 of the microneedle 300. The microneedle body 305 is pyramid shaped, however, it should be appreciated that depending on specific application requirements, other shapes are possible, for example, conical, hexagonal, etc. It will be appreciated that a geometry of the extended tip portion 304 can be substantially the same or different from a geometry of the body 305. The extended tip portion 304 can be of other dimensions, for example, less than 50 μm if required, depending on design requirements.

The microneedles described above can be made from polymers, for example, various grades of polycarbonate (PC), polystyrene (PS), and particularly bio-compatible polymers such as polyetherimide (PEI), polyetherehterketone (PEEK), etc. The type of polymer used to make the microneedle is chosen based on characteristic mechanical properties of the polymer that are suitable for specific dimensions of the microneedle designed for specific applications. In addition, each polymer can be filled with additives for the purposes of reinforcement and/or conductivity of the microneedle.

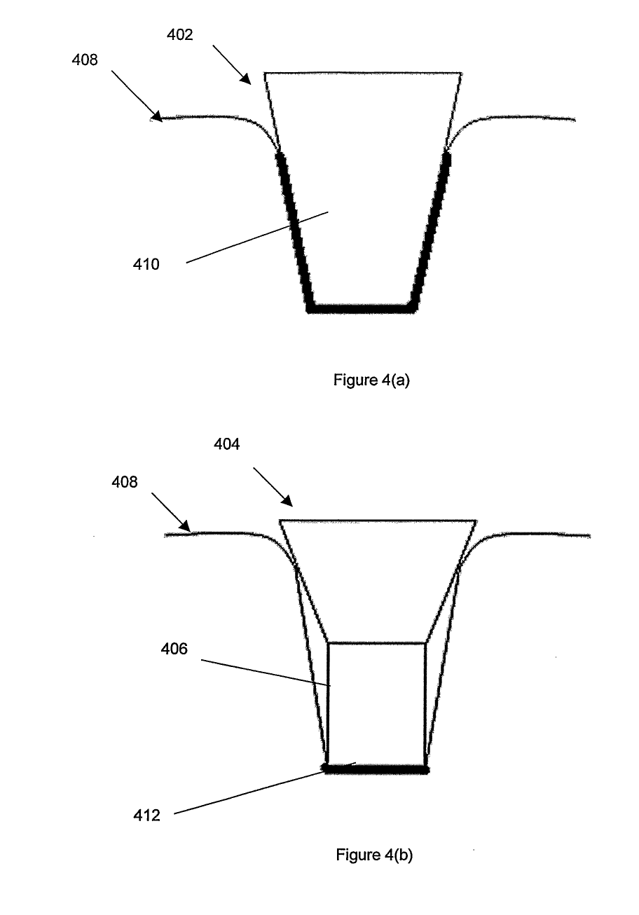

FIGS. 4(a) and 4(b) are schematic drawings of a portion of a microneedle 402 without an extended tip portion and a microneedle 404 with an extended tip portion 406, used to breach the stratum corneum (SC) 408 of skin. The side ports of the microneedles 402, 404 are not shown in FIGS. 4(a) and 4(b). The extended tip portion 406 of the microneedle 304 in FIG. 4(b) has the same width as the tip 410 of the microneedle 402 without the extended tip portion. Due to the highly deformable nature of skin (which is also viscoelastic), initial contact with the tip 410 of the microneedle 402 normally deforms and tensions the skin for a signification portion of an advancing stroke of the microneedle 402 before breach of the SC 408 occurs. During this deformation stage, the skin is forced to conform to the shape of the microneedle 402, particularly to the geometry of the tip 410. Therefore, comparing the microneedle 402 without the extended tip and the microneedle 404 with the extended tip portion 406, the microneedle 402 with a “normal” tip (i.e. without any extended tip portion) causes full conformance of the skin to the geometry of the tip 410. This in turn results in a larger effective contact area between the skin and the microneedle, as shown in FIG. 4(a). The effective contact areas are indicated by the bold lines in FIGS. 4(a) and 4(b). Since a threshold pressure must be reached before any puncturing or breach of the SC 408 can occur, a larger contact area reduces the resultant pressure caused by the tip 410 of the microneedle 402 on the skin and makes breaching of the SC 408 more difficult as more force has to be applied. However, for the microneedle 404 with the extended tip portion 406, the effective contact area between the skin and the microneedle 404 is restricted to the apex 412 of the extended tip portion 406 which has a smaller area compared to the effective contact area in FIG. 4(a). As a result of the reduced effective contact area, a higher pressure is exerted on the skin by the microneedle 404 and less force is applied to breach the SC 408.

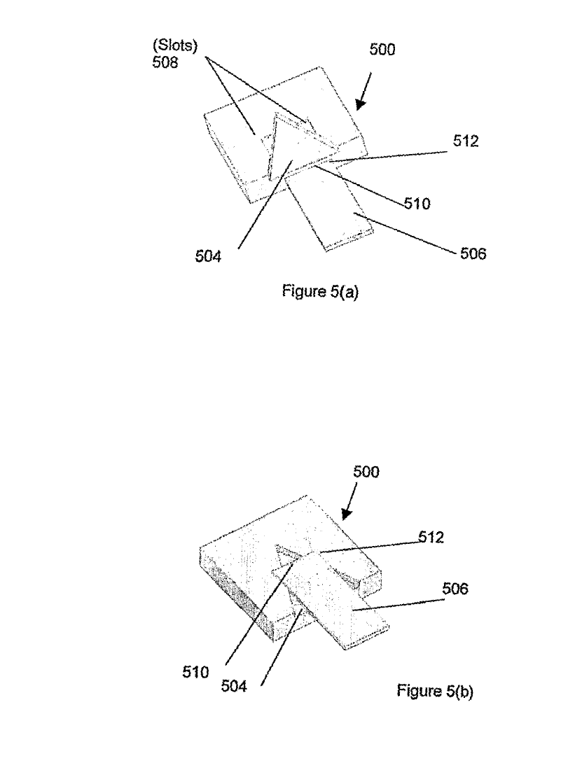

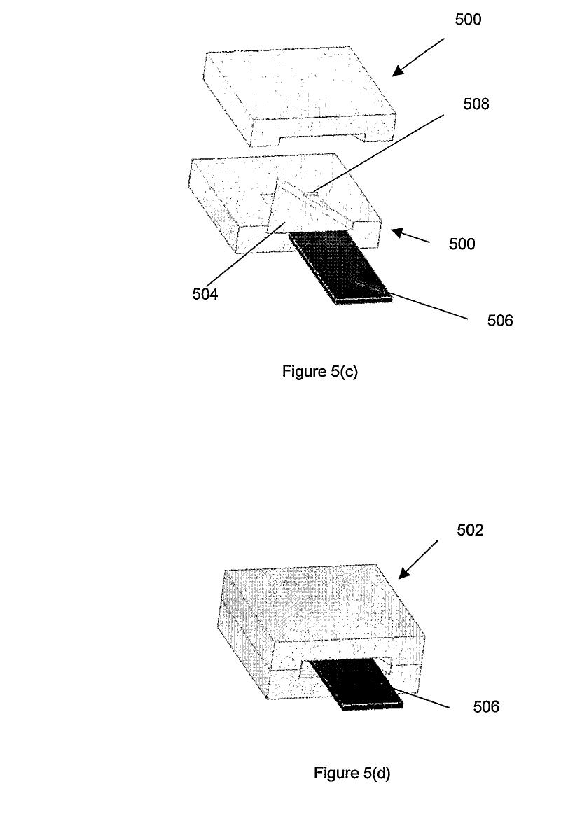

Schematic drawings of a mould 502 (FIG. 5(d)) used for fabricating a microneedle are shown in FIGS. 5(a)-5(d). The mould 502 comprises two symmetrical mould halves 500. Therefore, only one mould halve 500 is described. The mould halve 500 in FIGS. 5(a)-5(d) can be used to fabricate, for example, the microneedle 100 in FIGS. 1(a) and 1(b). The mould halve 500 comprises a cavity 504 defining a periphery of a negative shape of the microneedle to be fabricated, (e.g. microneedle 100 in FIG. 1(a)), an insert 506, and two slots 508 for aligning the insert 506 with reference to the cavity 504. The cavity 504 is triangular in shape and the insert 506 is rectangular shaped pin. When the pin 506 is inserted into the mould halve 500, as shown in FIG. 5(b), the pin 506 is aligned with the cavity 504 such that a leading edge 510 of the pin 506, in this case, leading corners 512 of the rectangular pin 506, intersect with the periphery of the cavity 504 at two locations (i.e. at two opposing walls defining the cavity) corresponding to, for example, the side ports 108 of the microneedle 100 in FIG. 1(a). Accordingly, the two slots 508 are shaped to align and hold the pin 506 in position within the mould halve 502. The shape of the slots 508 corresponds to the leading corners 512 of the rectangular pin 506 to align and hold the pin 506 within the mould 502.

To mould the microneedle, injection moulding, for example, is used to fill the mould 502 with molten polymer. Other types of moulding can also be used, for example, cast moulding. The two mould halves 500 are closed and held tightly together before the pin 506 is inserted into the mould 502 and aligned with respect to the mould cavity 504, as shown in FIGS. 5(c) and 5(d). A portion of the pin 506 remains outside the mould 502. The mould cavity 504 is then filled with a polymer melt, for example, by using injection moulding, to form the microneedle. The polymer melt (not shown) is injected into the mould 502 with an appropriate pressure to substantially fill the mould cavity 504 in the mould 502. The molten polymer will flow around the pin 506 such that portions of the mould cavity 504 occupied by the pin 506 is not filled with the polymer melt, thereby creating the lumen 110 of the microneedle 100 of FIG. 1(a), while the intersection of the leading edge 510 of the pin 506 with the periphery of the mould cavity 504 creates the side ports 108 of the microneedle 100 of FIG. 1(a). Therefore, by using the above fabrication method, microneedles with the lumen in fluid connection with the two side ports can be fabricated.

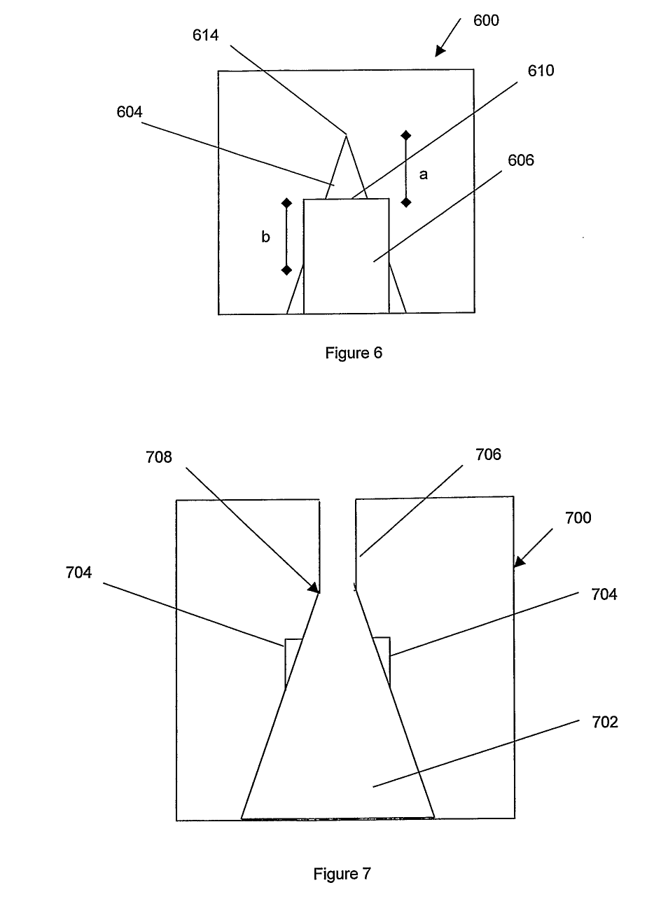

A schematic drawing of a portion of a mould halve 600, for example, the mould halve 500 of FIG. 5(a), is shown in FIG. 6. The submerge distance, a, of the microneedle (not shown in FIG. 6) is determined by the distance from a tip 614 of the cavity 604 to the intersection of the leading edge 610 of the pin 606 with the periphery of the cavity 604, while the extent to which the pin 606 intersects the periphery of the cavity 604 (i.e. distance b in FIG. 6) determines the size of the side ports (not shown) in the microneedle to be fabricated.

It will be appreciated that the shape of the cavity in the mould depends on the shape of the microneedle to be fabricated and can be of various shapes other than triangular. If only one dispensing port is required in the microneedle, then the leading edge of the pin can be made to only intersect with the periphery of the cavity at one location. The number of side-ports created in the microneedle therefore depends on the number of locations where the leading edge of the pin intersects with the periphery of the mould cavity. This can be achieved by different combinations of the shape of the mould cavity and the shape of the pin. For example, a conical mould cavity with a hexagonal pin will have six ports in a conical-shaped microneedle, while a hexagonal pyramid cavity with a circular pin will also have six dispensing ports in a tapered polygonal microneedle. The pin can be of other shapes, for example, cylindrical, polygonal, etc., instead of rectangular, depending on design requirements. Accordingly, depending on the geometry of the pin, the slot can be round or polygonal, etc.

It will be appreciated that the mould 502 or mould halves 500 of FIGS. 5(a) to 5(d) can, for example, be modified to comprise a plurality of cavities 504 arranged side by side. Furthermore, a plurality of moulds 502 can, for example, be stacked onto one another to fabricate a plurality of microneedles or an array of microneedles.

A schematic drawing of a cross section of another mould halve 700 used for fabricating a microneedle is shown in FIG. 7. The mould halve 700 in FIG. 7 can be used to fabricate, for example, the microneedle 300 in FIGS. 3(a) and 3(b). It will be appreciated that a mould (not shown in FIG. 7) comprising two symmetrical mould halves 700 is used for fabricating the microneedle. Therefore, only one mould halve 700 is shown and described.

The mould cavity 702 comprises two slots 704 for aligning and holding a pin (not shown in FIG. 7), and a vent 706 configured for creating the extended tip portion 304 of the microneedle 300 in FIGS. 3(a) and 3(b). The vent 706 is generally an elongate channel extending from an apex 708 of the mould cavity 702.

The microneedle can be moulded by, for example, using injection moulding to fill the mould cavity 702 with polymer melt. A polymer melt (not shown) is injected into the mould with an appropriate pressure to substantially fill the mould cavity 702 in the mould.

Polymer melt is generally viscous and therefore has difficulty filling mould cavities. This becomes a significant problem when moulding objects of small dimensions, for example, microneedles, in particular the tips or portions near the tips of the microneedles where the dimensions can be about 10-100 microns in size. Air in the microneedle cavities is pushed by the polymer melt during moulding and trapped in the microneedle cavity forming voids in the moulded object. For example, air can be trapped near or at the tips of the microneedle cavities, causing the tips in the resulting microneedles to be less sharp and not well defined.

By using a mould with a vent, for example, the vent 706 in FIG. 7, as the polymer melt fills the mould cavity 702, air trapped in the mould cavity 702 is vented out of the mould via the vent 706. When the mould cavity 702 is substantially filled with the polymer melt, additional pressure is applied to the polymer melt such that the polymer melt partially fills the vent 706 to form the extended tip portion 304 of the microneedles 300 in FIG. 3(a). Therefore, the vent 706 is configured for creating the extended tip portion 304 of the microneedle 300 in addition to provide for venting of air out of the mould cavity 702. The length of the extended tip portion 304 of the microneedle 300 is accordingly determined by the level of polymer melt filling the vent 706. Injection moulding can be carried out in a vacuum chamber to remove any residual air trapped in the mould cavities and to assist the polymer melt in entering and filling the microneedle cavities. Further, a vacuum oven can be used to melt the polymer.

It will be appreciated that the mould halves 500 shown in FIGS. 5(a)-5(d) can also comprise a vent formed near or at an apex of the mould cavity 504 as described above for venting of air out of the mould cavity 504. In order to create microneedles without the extended tip, for example, the microneedle 100 in FIGS. 1(a) and 1(b) or the microneedle 200 in FIG. 2, an appropriate pressure is applied to the polymer melt during the injection molding process such that the polymer melt does not fill the vent.

In FIGS. 5(a) to 5(d) and FIG. 6 and FIG. 7, the pin 506, 606 is a rectangular insert. It will be appreciated that the pin can have different geometries, depending on design requirements. The shape of lumen created will depend on the geometry of the pin. In general, the lumen defines a periphery of a negative shape of the pin. The pin may comprise different geometry and/or dimension at a stem portion and at the leading edge, for example, when a larger lumen is required to reduce dispensing pressure of the drug and a particular size of side-ports are required. Alternatively, the pin can be made tapered.

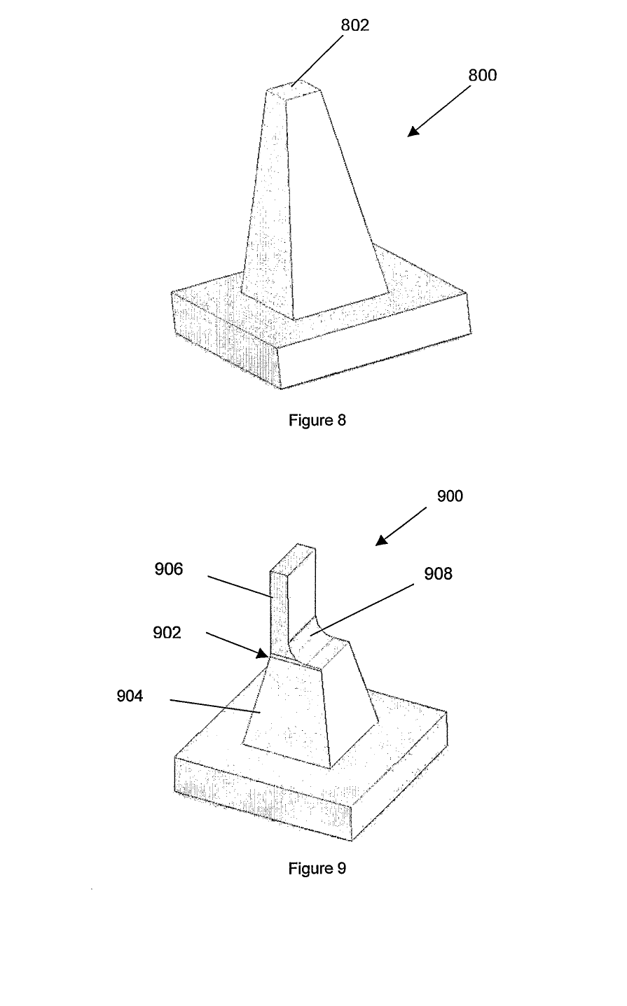

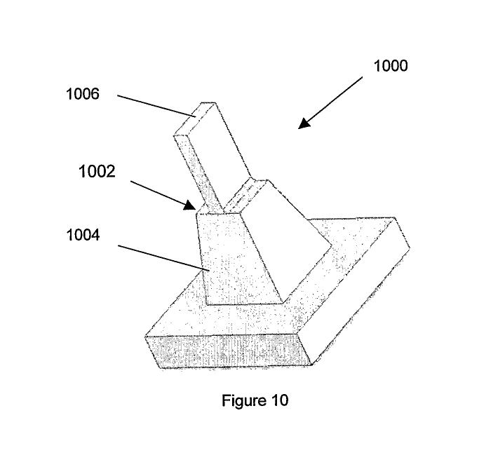

A schematic drawing of an isometric view of another pin 800 is shown in FIG. 8. The pin 800 is substantially pyramid in shape with a flat apex portion 802.

A schematic drawing of an isometric view of another pin 900 is shown in FIG. 9. The pin 900 comprises a body 902 having a first section 904 and a second section 906. The first section 904 tapers towards the second section 906. The second section 906 is a rectangular block extending from the first section 904. The first section 904 is connected to the second section 906 by a chamfered portion 908.

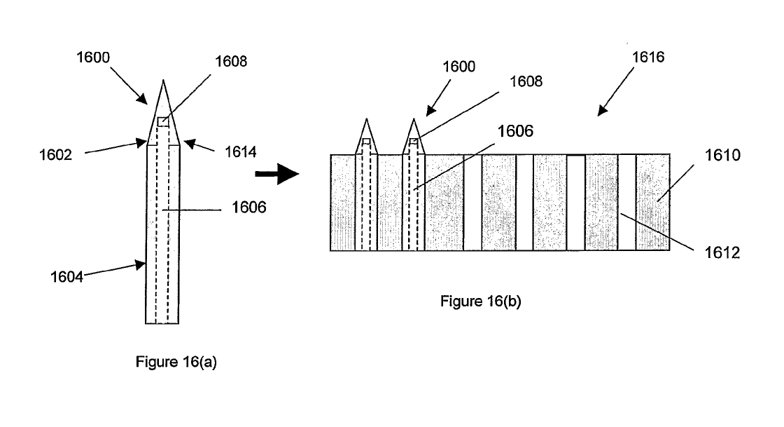

A schematic drawing of an isometric view of another pin 1000 is shown in FIG. 16. The pin 1000 comprises a body 1002 having a first section 1004 and a second section 1006. The first section 1004 tapers towards an intermediate surface 1008 between the first section 1004 and the second section 1006. The second section 1006 is a rectangular block extending from the middle of the intermediate surface 1008.

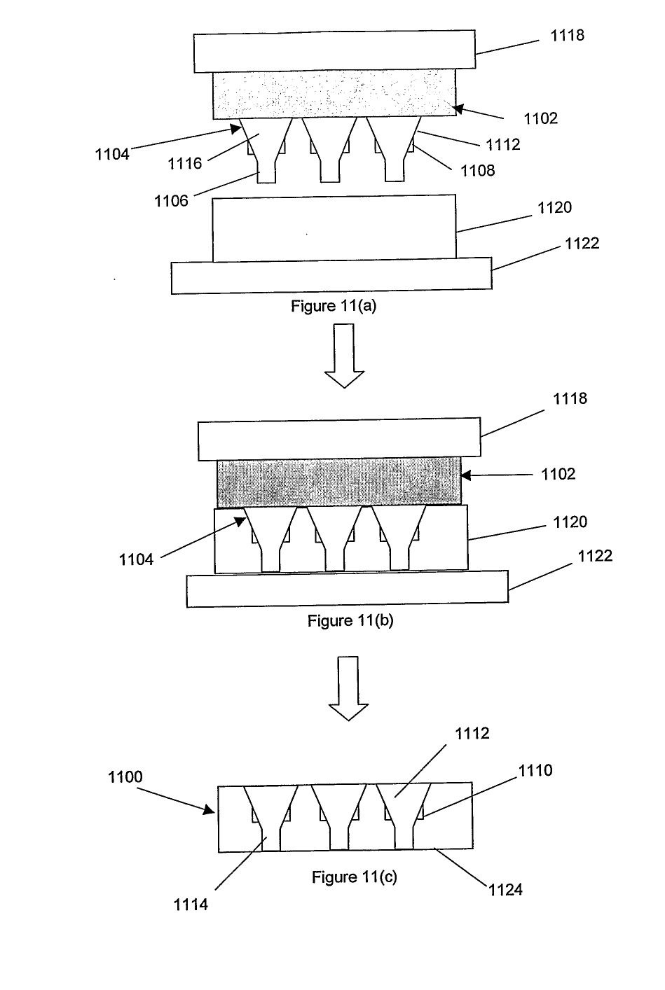

As described above, the mould (e.g. the mould 502 in FIG. 5(d) or mould 700 FIG. 7) used to fabricate the microneedles comprises cavities that generally define a periphery of a negative shape of the microneedles to be fabricated. One way of creating the mould is to first create a master mould comprising a positive shape of the microneedles. In other words, the master mould comprises protrusions that are generally corresponding to the shape of the microneedles that are to be fabricated. The protrusions on the master mould can be fabricated by precision wire-cutting or other precision engineering means. The pattern of the protrusions corresponding to the shape of the microneedles on the master mould is then transferred onto a secondary mould by, for example, hot embossing, to form cavities corresponding to a negative shape of the microneedles. Schematic drawings of a process for making a secondary mould 1100 for use in fabricating microneedles are shown in FIGS. 11(a)-11(c). The secondary mould 1100, can be in the form of for example, the mould 700 in FIG. 7, and can be used to form, for example, the microneedle 300 of FIG. 3(a). The master mould 1102 comprises a plurality of protrusions 1104 corresponding to the shape of the microneedles (e.g. microneedle 300 of FIG. 3(a)) to be fabricated is first created. The master mould 1102 is made of tool steel. The protrusions 1104 on the master mould 1102 can be made by precision wire cutting. Generally, the shape of the protrusions 1104 on the master mould 1102 are positive images of the microneedles to be fabricated. The protrusions 1104 are pyramid shaped with extended tip portions 1106. The extended tip portions 1106 of the protrusions 1104 are used to form vents 1114 in the secondary mould 1100. Each protrusion 1104 comprises two triangular shaped shoulder portions 1108 protruding from two opposing sidewalls 1114 of a body portion 1116 of the protrusions 1104. The two triangular shaped shoulder portions 1108 are used to form the slots 1110 in the cavity 1112 of the secondary mould 1100. It will be appreciated that the shoulder portions 1116 can be of other geometries depending on the shape of the slot 1110 that is required. Generally, a shape of the shoulder portions 1116 is a positive shape of the slots 1110 in the secondary mould 1100. In this case, since the pin (not shown) is rectangular in shape, the shape of the slots 1110 are triangular in shape and therefore, the shoulder portions 1116 are also triangular in shape.

The master mould 1102 mounted to a first heated platen 1118 is hot embossed into a metal substrate 1120 mounted on a second heated platen 1122 to form a negative image of the master mould 1102 (and therefore a negative image of the microneedles) in the substrate 1120 to form the secondary mould 1100 that is used for fabricating the microneedles. A base portion 1124 of the secondary mould 1100 is removed, for example, by grinding, such that the vents 1114 extend through the secondary mould 1100. It will be appreciated that alternatively, the extended tip portions 1106 of the master mould 1100 can be made longer such that the vents 1114 extend through the base portion 1124 of the substrate 1120 during hot embossing (i.e. no grinding of the base portion 1124 is required).

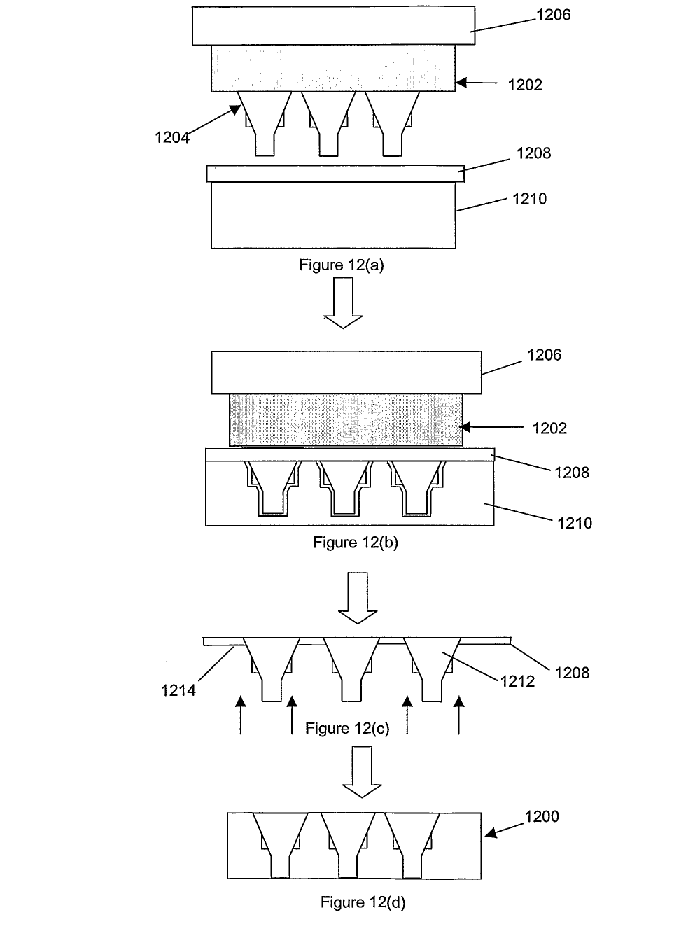

Schematic drawings of another process for making a secondary mould 1200 for use in fabricating microneedles are shown in FIGS. 12(a)-12(d). The secondary mould 1100, can be in the form of for example, the mould 700 in FIG. 7, and can be used to form, for example, the microneedle 300 of FIG. 3(a). In this example, microforming is used to fabricate the microneedles. The master mould 1202 comprising the protrusions 1204 is mounted to a punch 1206. A metal sheet 1208 is placed on a deformable die 1210 such that the metal sheet 1208 is between the punch 1206 and the deformable die 1210. The metal sheet 1208 can be made of metallic materials such as steel, aluminium and copper. The thickness of the metal sheet 1208 can range from about 0.05 mm to about 0.5 mm. However, other dimensions, for example, a thickness of greater than 0.5 mm can also be used depending on design requirements. The punch 1206 together with the master mould 1202 is moved towards the metal sheet 1208 and presses against the metal sheet 1208 and the deformable die 1210. The metal sheet 1208 is deformed and a negative shape of the protrusions 1204 of the master mould 1202 is created onto the metal sheet 1208. Therefore, the metal sheet 1208 comprises cavities 1212 generally defining the periphery of the negative shape of the microneedles to be formed. At the same time, the deformable die 1210 is also deformed by the protrusions 1204 of the master mould 1202 to form cavities defining the negative shape of the protrusions 1204. The punch 1204 together with the master mould 1202 is then retracted. The deformed metal sheet 1208 in FIG. 12(b) is removed and a thickness reinforcement process, for example, metal deposition is performed on a surface 1214 of the metal sheet 1208 defining an exterior surface of the cavities 1212 to form the secondary mould 1200 used for fabricating the microneedles. The direction of metal deposition is represented by the arrows in FIG. 12(c).

Other methods of fabricating the secondary mould are also possible, for example, by first fabricating an intermediate mould using the master mould and subsequently fabricating the secondary mould using the intermediate mould.

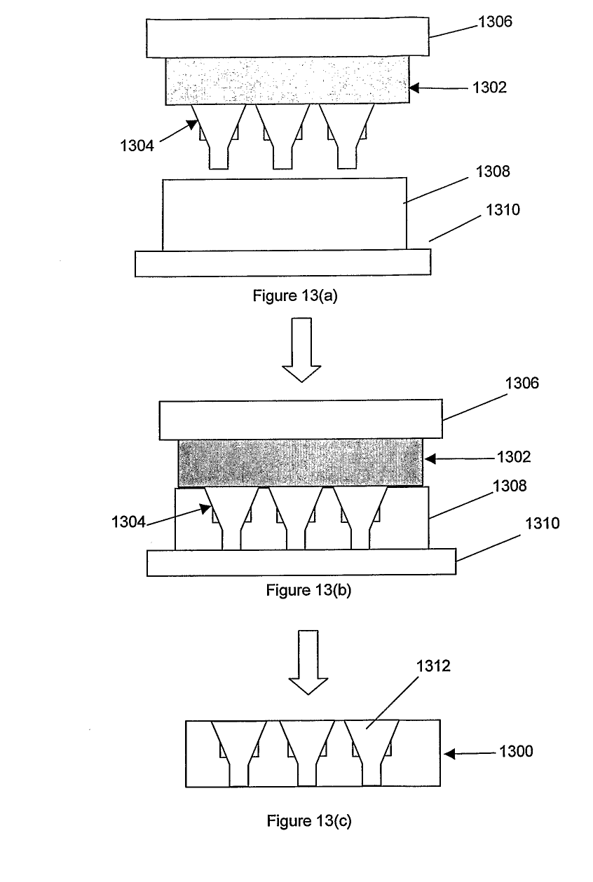

Schematic drawings of a process for making an intermediate mould 1300 are shown in FIGS. 13(a)-13(c). As described earlier, the master mould 1302 comprises protrusions 1304 that are generally corresponding to the shape of the microneedles that are to be fabricated. The protrusions 1304 on the master mould 1304 is made of tool steel and can be fabricated by precision wire-cutting or other precision engineering means. The master mould 1302 mounted to a first heated platen 1306 is hot embossed into a polymer substrate 1308 mounted on a second heated platen 1310 to form a negative image of the protrusions 1304 of the master mould 1302 (and therefore a negative image of the microneedles) in the polymer substrate 1308. The resulting embossed polymer substrate 1300 (i.e. the intermediate mould) comprising cavities 1312 defining a negative image of the protrusions (and therefore, a negative image of the microneedles) is then used to fabricate the secondary mould (not shown in FIGS. 13(a) to 13(c)) for fabricating microneedles.

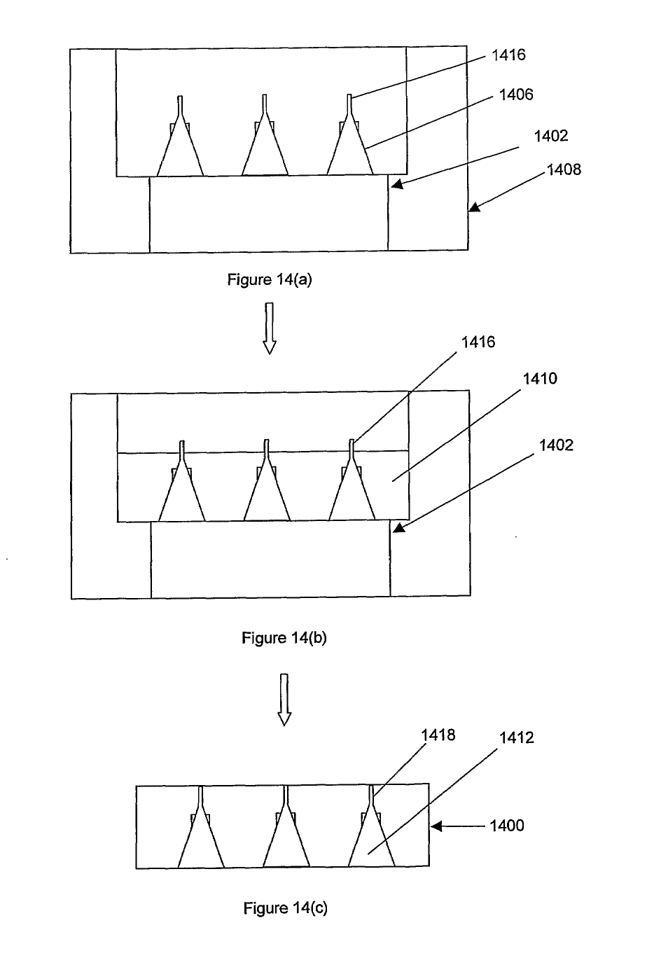

Schematic drawings of another process for making an intermediate mould 1400 are shown in FIGS. 14(a)-14(c). In this example process, the intermediate mould 1400 is cast moulded. The master mould 1402 comprising the protrusions 1406 forms part of a casting mould 1408 used in the casting process. Molten polymer 1410 into the casting mould 1408 and over the master mould 1402 as shown in FIG. 14(b). The molten polymer 1410 is allowed to cure and removed from the casting mould 1408 to obtain the intermediate mould 1400 comprising cavities 1412 defining a negative shape of the microneedles to be fabricated. In this example, the protrusions 1406 are pyramid shaped and each protrusion 1406 includes an extended tip portion 1416 for forming an air vent 1418 in the intermediate secondary polymeric mould 1400. The length of the vent 1418 of the intermediate mould 1400 is determined by the level of the molten polymer 1410 filling the casting mould 1408 (see FIG. 14(b)).

The polymeric intermediate moulds described in FIGS. 13(a)-13(c) and FIGS. 14(a) to 14(c) may be made of various types of polymers such as polycarbonate (PC), polystyrene (PS), polyetherimide (PEI), polypropylene (PP), polyethylene (PE), etc. in sheet form or poly(dimethylsiloxane) (PDMS) in liquid form for cast moulding.

In the embodiments described above, the polymer used for making the secondary mould is chosen to have a certain elasticity and deformability characteristic such that the secondary moulds can be re-used. A mould release agent can be applied or sprayed onto the secondary mould before polymer melt is poured into the secondary mould to form microneedles. This allows effective release of the moulded plastic microneedles and at the same time preserves the secondary mould from damage to allow subsequent use.

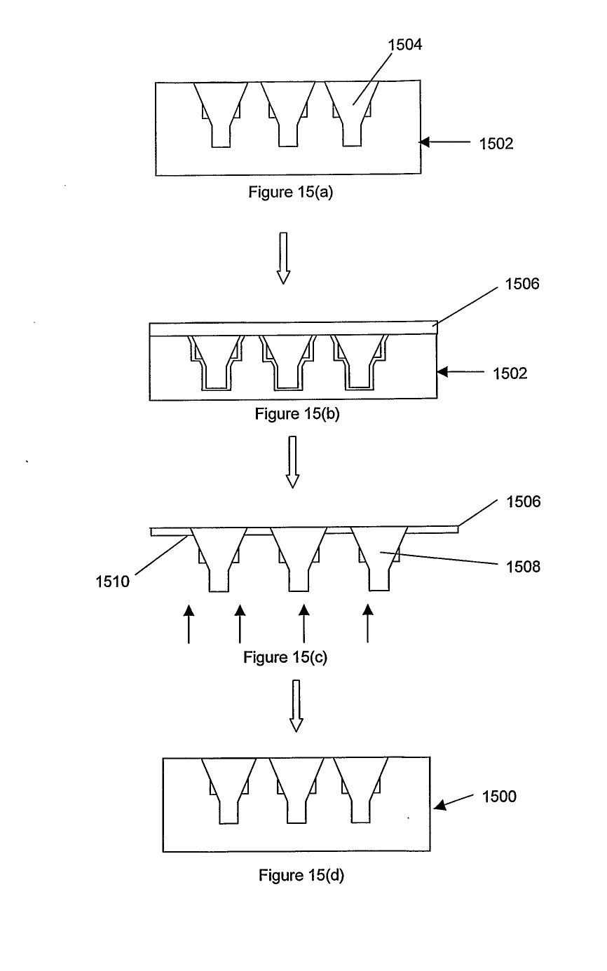

Schematic drawings of a process for making a secondary mould 1500 using an intermediate mould 1502 are shown in FIGS. 15(a)-15(d). The intermediate mould 1502 can, for example, be in the form of the intermediate mould 1300 in FIGS. 13(a)-13(c) or the intermediate mould 1400 in FIGS. 14(a)-14(c). As described above, the intermediate mould 1502 comprises cavities 1504 defining a negative shape of the microneedles to be fabricated. A layer of metal 1506 is deposited onto the intermediate polymeric secondary mould 1502 and into the cavities 1504 of the intermediate polymeric secondary mould 1502 (e.g. by chemical or vapour deposition, electroforming, electroplating, etc.). The deposited layer of metal 1506 comprising cavities 1508 is removed from the intermediate polymeric secondary mould 1502 as shown in FIG. 15(c). The deposited layer of metal 1506 is subjected to a thickness reinforcement process, for example, metal deposition performed on a surface 1510 of the deposited metal layer 1506 defining an exterior surface of the cavities 1208 to form the secondary mould 1500 used for fabricating the microneedles. The direction of metal deposition is represented by the arrows in FIG. 15(c).

A schematic drawing of another microneedle 1600 is shown in FIG. 16(a). The microneedle 1600 comprises a tip portion 1602, a stem portion 1604 comprising a lumen 1606. The stem portion 1604 is generally elongate structure extending from a base portion 1614 of the tip portion 1602. The stem portion 1604 has a substantially similar width as a base 1614 of the tip portion 1602. It will be appreciated that the stem portion 1604 can have a width that is larger or smaller than the base of the tip portion, depending on design requirements. The tip portion 1602 comprises a side port 1608. The side port 1608 extends into the lumen 1606 such that the side port 1608 and the lumen 1606 are in fluid communication. It will be appreciated that the microneedle 1600 can comprise the various features described in the earlier examples, for example the extended tip portion 304 of the microneedle 300 of FIG. 3(a).

FIG. 16(b) shows a plurality of microneedles 1600 assembled onto a platform 1610 to form a microneedle module 1916. The platform 1610 comprises a plurality of bores 1612. Each microneedle 1600 can be assembled into the bore 1612 such that the lumen 1606 of the microneedle 1600 is in fluid connection with a common reservoir (not shown) or to an individual reservoir (not shown) connected to the bore 1612 for dispensing or extracting fluid. The microneedles 1600 may e.g. be fabricated using a mould structure similar to the one described above with reference to FIGS. 5(a) to 5(d).

It will be appreciated that an array of microneedles 1600 can be obtained depending on the arrangement of bores 1612 on the platform 1610. For example, if the bores 1612 are arranged in a two dimensional array, a two-dimensional array of microneedles 1600 can be obtained. Further, individual microneedles 1600 can be fabricated separately from the platform 1610. This allows greater flexibility in the design of the arrays as well as provides more choices of materials that can be used for the platform and/or the microneedles compared with a plurality of microneedles that are fabricated integrally onto a substrate base. In the latter array, a new mould has to be created for different array designs.

It will be appreciated that the microneedles described in the above embodiments can be fabricated in an array. Microneedle arrays can e.g. be about 2-10 cm².

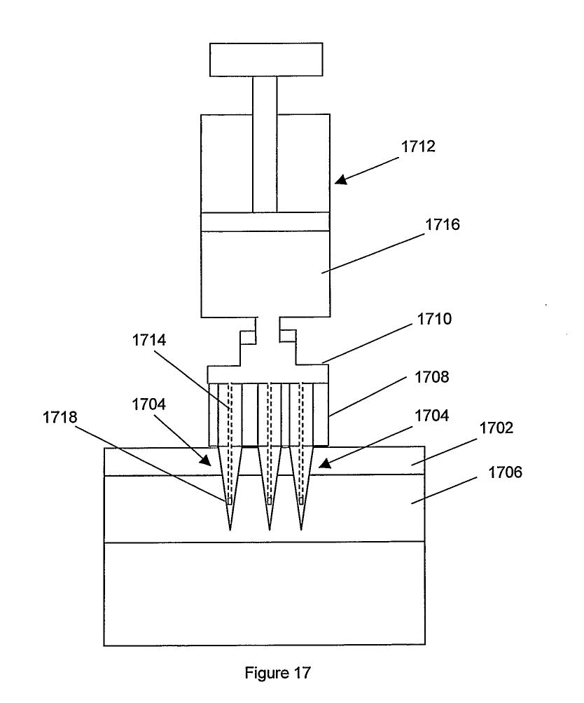

FIG. 17 is a schematic drawing illustrating a transdermal drug delivery mode. In the transdermal delivery mode a drug (or a liquid) is administered into a body across the skin, diffusing into blood capillaries (not shown). The outermost layer of the skin which is called stratum corneum (SC) 1702 comprises dead cells forming a water-proof layer that hinders the transportation of drugs from outside the body to inside the body. There are several methods to overcome the barrier presented by the SC 1702. One example is to use microneedles 1704 to breach the SC 1702 and deliver drug shallowly into the skin by puncturing the SC 1702 and inserted within an epidermis layer 1706. Theoretically this method of drug delivery should incur minimal pain, if not be painless. The microneedles 1704 are assembled on a platform 1708 and can be in the form of e.g. the microneedle module 1612 in FIG. 16(b). The platform 1708, can be received in an adaptor 1710 for coupling to a syringe 1712 such that the lumen 1714 of each microneedle 1704 is in fluid communication with a reservoir 1716 within the syringe 1712. During drug delivery, drugs stored in the reservoir 1716 is dispensed into epidermis layer 1706 of the skin via the side ports 1718.

The microneedles 1704 can also be used for extracting body fluid, which may include whole blood sampling. Body fluid can be extracted from the body, e.g. from the blood capillaries, via the side ports 1718 and the lumen 1714 for whole blood sampling. The extracted body fluid can be stored in the reservoir 1716 in the syringe 1712.

The platform 1708 and the adaptor 1710 can be made of plastic.

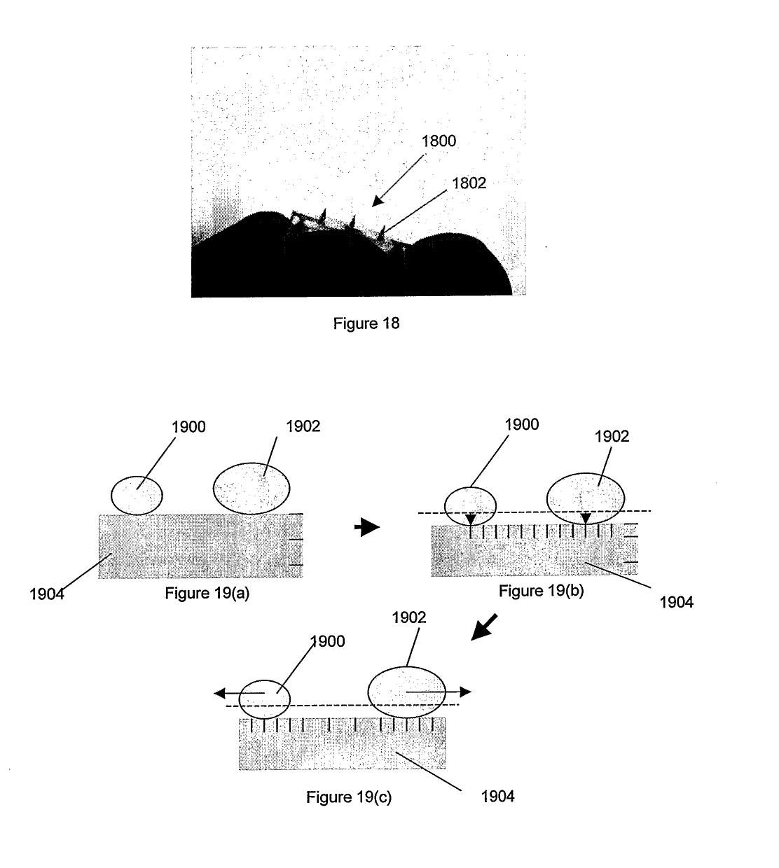

A micrograph of an array 1800 of microneedles 1802 having side ports and an extended tip portion is shown in FIG. 18.

Skin tensioning and compression may be required prior to the breaching of the SC to increase penetration effectiveness. This can be achieved, for example, by using a middle 1900 finger and a thumb 1902 to compress the skin (FIG. 19(b)) and tension the skin 1904 (FIG. 19(c)), and pressing the skin with the microneedle platform using appropriate force, as shown in FIGS. 19(a) to 19(c).

The microneedles can be made about 0.1-1.0 mm long on a platform of several mm thick for the purpose of breaching the SC. The diameter of the larger end of the body of the microneedles may be several times smaller than the length, for example, the base dimension of a 750-micron long microneedle can be 300 microns. The pitch between two microneedles is similar to the length of the microneedle, and in some cases slightly smaller that the length. The area of the array can vary from about 2-10 cm². The breaching of the SC by the microneedle array may be assisted by an external applicator or by hand.

The microneedles described are designed to minimally puncture the skin by control of the length (penetration depth), size and sharpness so as to reduce trauma.

Conventional transdermal delivery means such as iontophoresis, electrophoresis, electroporation, sonophoresis (ultrasound), etc. targets to breach the SC by non-invasive means, but these techniques have not been able to show sufficient reproducibility and convincing clinical results, therefore there is still great reliance on mechanically breaching the SC for transdermal delivery.

A microneedle array can be used to puncture the skin by pressing the microneedle platform onto the skin. Subsequently the platform is removed from the puncture site and is disposed of. A patch coated with a bio-active substance may be affixed onto the puncture site to allow passive diffusion of drug into the skin while isolating the breached skin from the external environment.

Further, an array of microneedles can be arranged on a flexible platform and incorporated as a cosmetic pad used for skin care purposes.

It will be appreciated by a person skilled in the art that numerous variations and/or modifications may be made to the present invention as shown in the specific embodiments without departing from the spirit or scope of the invention as broadly described. The present embodiments are, therefore, to be considered in all respects to be illustrative and not restrictive.

1. A plastic microneedle comprising:
a body portion tapering from a larger end of the body portion towards a tip portion of the body portion;
at least one side port formed in the body portion; and
a lumen extending from the larger end of the body portion and within the body portion of the microneedle, wherein the side port extends into the lumen such that the side port and the lumen are in fluid communication with each other and such that a fluid discharge direction from the side port is inclined with reference to a longitudinal direction of the lumen.

2. The microneedle as claimed in claim 1, wherein the side port is disposed on a side surface of the body portion of the microneedle.

3. The microneedle as claimed in claim 1, comprising two side ports disposed at opposing side surfaces of the body portion of the microneedle.

4. The microneedle as claimed in claim 1 further comprising an extended tip portion formed on the tip portion of the body portion and forming an apex of the microneedle, wherein the extended tip portion comprises a substantially elongate protrusion extending from the tip portion.

5. The microneedle as claimed in claim 4, wherein a geometry of the extended tip portion is different from a geometry of the body portion.

6. The microneedle as claimed in claim 1 wherein the microneedle is made from at least one of a group of polymers consisting of polycarbonate (PC), polystyrene (PS), polyetherimide (PEI), polyetherehterketone (PEEK).

7. The microneedle as claimed in claim 1, wherein the body portion of the microneedle is pyramid shaped, conical shaped, or hexagonal shaped.

8. The microneedle as claimed in claim 1 are fabricated using injection moulding or cast moulding.

9. A platform member comprising an array of microneedles as claimed in claim 1.

10. A method of fabricating a plastic microneedle, the method comprising:
forming a body portion, the body portion tapering from a larger end of the body portion a tip portion of the body portion;
forming at least one side port in the body portion; and
forming a lumen extending from the larger end of the body portion and within the body portion of the microneedle, wherein the side port extends into the lumen such that the side port and the lumen are in fluid communication with each other and such that a fluid discharge direction from the side port is inclined with reference to a longitudinal direction of the lumen.

11. A method as claimed in claim 10 further comprising:
forming an extended tip portion on the tip portion of the body portion and the extended tip portion forming an apex of the microneedle, wherein the extended tip portion comprises a substantially elongate protrusion extending from the tip portion.

12. A method as claimed in claim 10, the method comprising:
forming a master mould comprising a positive shape of the microneedle,
using the master mould to form a secondary mould comprising a cavity comprising a periphery of a negative shape of the microneedle; and
using the secondary mould to form the microneedle.

13. A method as claimed in claim 10, the method comprising:
forming a master mould comprising a positive shape of the microneedle,
using the master mould to form an intermediate mould comprising a cavity comprising a periphery of a negative shape of the microneedle; and
using the intermediate mould to form a secondary mould comprising a cavity defining the periphery of a negative shape of the microneedle.

14. A method as claimed in claim 13, wherein the intermediate mould is made of polymer.

15. A method as claimed in claim 12, wherein the positive shape of the microneedle on the master mould is created by precision wire-cutting.

16. A method as claimed in claim 12, wherein the secondary mould is made of metal.

17. A method as claimed in claim 12, further comprising: filling the cavity of the secondary mould with a polymer to form the microneedle.

18. A method as claimed in claim 12, the method further comprising:
providing an insert member;
providing the secondary mould, the secondary mould further comprising at least one slot portion for receiving and aligning the insert member such that a leading edge of the insert intersects the periphery of the negative shape of the microneedle;
inserting the insert member into the secondary mould; and
filling the secondary mould with a fill material, wherein
the insert creates the lumen in the microneedle and the intersection of the leading edge of the insert with the periphery of the negative shape of the microneedle creates the side port extending into the lumen such that the lumen and the side port are in fluid communication with each other.

19. The method of fabricating microneedles as claimed in claim 18, wherein the periphery of the negative shape of the microneedle comprises a channel extending from an apex of the negative shape of the microneedle for forming the extended tip portion of the microneedle.

20. The method of fabricating microneedles as claimed in claim 19, further comprising filling the channel at least partially to form the extended tip portion.

21. A use of a microneedle as claimed in claim 1 for injecting liquid into a body.

22. A use of a microneedle as claimed in claim 1 for extracting body fluid from a body.

23. The use as claimed in claim 22, wherein extracting body fluid from the body includes whole blood sampling.

24. The microneedle as claimed in claim 4, wherein a geometry of the extended tip portion is substantially the same as a geometry of the body portion.
